(19) United States
(12) Patent Application Publication
(10) Pub. No.: US 2005/0148862 A1
(43) Pub. Date: Jul. 7, 2005

(54) GRADIENT COIL APPARATUS AND METHOD OF ASSEMBLY THEREOF

(75) Inventor: Michael Ben Sellers, Florence, SC (US)

Correspondence Address: Philmore H. Colburn II, Cantor Colburn LLP, 55 Griffin Road South, Bloomfield, CT, 06002, US

(73) Assignee: GE MEDICAL SYSTEMS GLOBAL TECHNOLOGY COMPANY, LLC, Waukesha, WI (US)

(21) Appl. No.: 10/707,907

(22) Filed: Jan. 23, 2004

(30) Foreign Application Priority Data
Dec. 22, 2003 (GB) 0329672.0

(51) Int. Cl. A61B 5/05

(52) U.S. Cl. 600/422

(57) ABSTRACT

A gradient coil assembly for use in an MRI device and a method for assembling the gradient coil assembly is provided. The gradient coil assembly includes a gradient tube extending along an axis. The tube includes first and second gradient coils and a conductive compound disposed between the first and second gradient coils.

### CROSS REFERENCE TO RELATED APPLICATIONS

This application claims the benefit of a priority under 35 U.S.C. 119 to Great Britain Patent Application No. 0329672.0 filed Dec. 22, 2003, the entire contents of which are hereby incorporated by reference.

### BACKGROUND OF INVENTION

The invention relates generally to a gradient coil assembly and a method for assembling the gradient coil assembly.

Magnetic Resonance Imaging (MRI) machines utilize gradient coils to generate magnetic fields along desired axes. Further, adjacent gradient coils are generally separated a predetermined distance. Further, a gap formed between first and second gradient coils is generally filled with a nonconductive epoxy resin. The inventor herein has recognized that air bubbles may be trapped within the nonconductive epoxy resin. During energization of first and second gradient coils, a voltage potential may be induced between a first end and a second end of each air bubble contained with the resin. When the voltage potential reaches a predetermined threshold voltage, an electrostatic discharge may be propagated across the air bubbles. The electrostatic discharge may generate undesirable bursts of electromagnetic radiation that can create a “snowy” image in an MRI machine.

Thus, the inventor herein has recognized that there is a need for minimizing a voltage potential induced in compounds, such as resins or glues, disposed between first and second gradient coils. By minimizing the induced voltage potential, the inventor has realized that undesirable electrostatic discharges can be minimized and/or eliminated.

### SUMMARY OF INVENTION

The foregoing problems and disadvantages are overcome by a gradient coil assembly and method for assembling the gradient coil assembly in accordance with the exemplary embodiments disclosed herein.

A gradient coil assembly in accordance with exemplary embodiments includes a a gradient tube extending along an axis. The tube includes first and second gradient coils and a conductive compound disposed between the first and second gradient coils.

A method for assembling a gradient coil assembly in accordance with exemplary embodiments is provided. The method includes disposing a first gradient coil on a first gradient tube. The method further includes disposing a conductive compound between the first gradient coil and a second gradient coil.

Other systems and/or methods according to the embodiments will be or become apparent to one with skill in the art upon review of the following drawings and detailed description. It is intended that at all such additional systems, methods, and/or computer program products be within the scope of the present invention, and be protected by the accompanying claims.

### BRIEF DESCRIPTION OF DRAWINGS

FIG. 1 is a block diagram of an MRI imaging system.

FIG. 2 is a cross-sectional view of a gradient tube assembly used in the MRI imaging system.

FIG. 3 is a flowchart of a method of assembling the gradient tube assembly.

### DETAILED DESCRIPTION

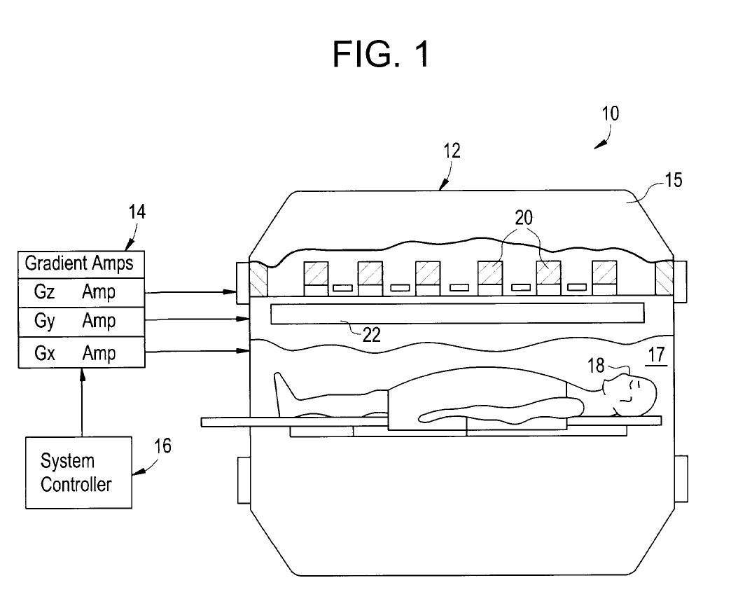

Referring to the drawings, identical reference numerals represent identical components in the various views. Referring to FIG. 1, an exemplary MRI system 10 is provided for generating images of a person 18. MRI system 10 may comprise a magnetic assembly 12, a gradient amplifier unit 14, and a system controller 16.

Magnetic assembly 12 is provided to generate magnetic fields that will be propagated to person 18. Assembly 12 may comprise a housing 15 defining a chamber 17 for receiving person 18. Assembly 12 may further comprise polarizing magnets 20, and a gradient coil assembly 22 having (X) coils, (Y) coils, and (Z) coils. Gradient coil assembly 22 generate magnetic fields along a predetermined X-axis, Y-axis, Z-axis in response to signals received from the (Gz) amplifier, (Gy) amplifier, and (Gz) amplifier, respectively, contained in gradient amplifier unit 14.

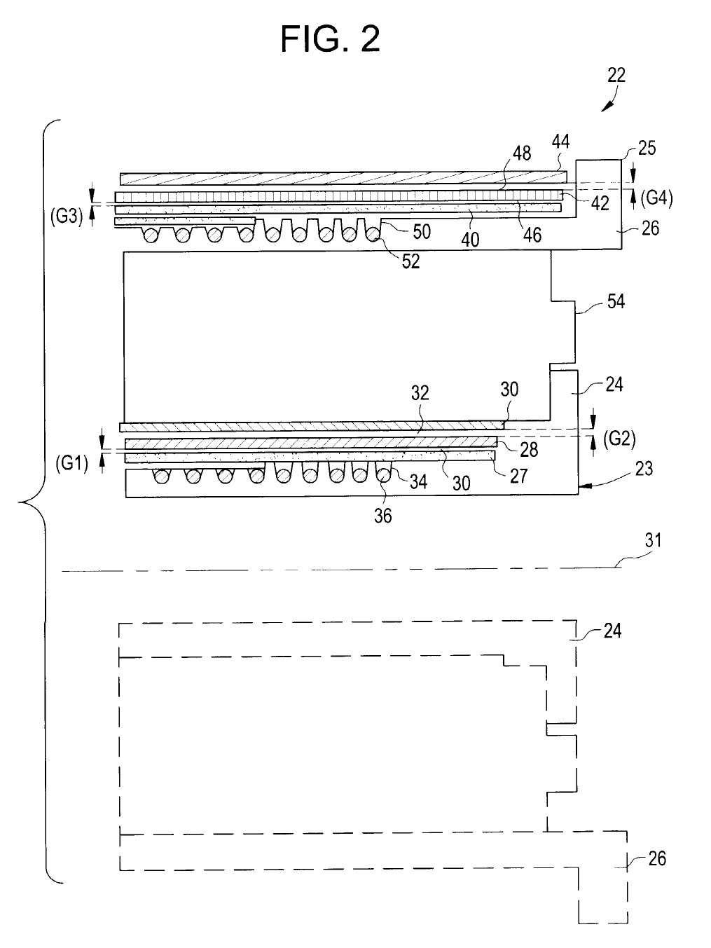

Referring to FIG. 2, an exemplary gradient coil assembly 22 includes an inner gradient coil assembly 23, an outer gradient coil assembly 25, and an epoxy layer 54 disposed between assemblies 23,25.

Inner gradient coil assembly 23 includes an inner gradient tube 24, an inner (Z) coil 27, an inner (Y) coil 28, an inner (X) coil 30, and conductive compound layers 27, 32.

Inner gradient tube 24 is provided to be disposed within an outer gradient tube 26 and is disposed about an axis 31. Gradient tube 24 may be constructed from a fiber composite material comprising one or more layers wherein each layer comprises a plurality of fibers such as glass fibers, carbon fibers, Kevlar fibers, and aluminum oxide particles, coated with the epoxy resin.

Inner (Z) coil 27 is provided to generate a magnetic field gradient along a predetermined Z-axis (not shown). Coil 27 may be disposed proximate gradient tube 24. In particular, coil 27 may be disposed on an outer surface of tube 24. Coil 27 may include a plurality of copper conductors 36 disposed in a plurality of grooves 34 formed in tube 24.

Inner (Y) coil 28 is provided to generate a magnetic field gradient along a predetermined Y-axis (not shown). Coil 28 may comprise a plurality a saddle coils (not shown). Coil 28 may be disposed a predetermined distance away from coil 27. In particular, a gap (G1) may be defined between coils 27, 28. A conductive compound layer 27 may be disposed in the gap (G1) which will be explained in greater detail below.

Inner (X) coil 30 is provided to generate a magnetic field gradient along a predetermined X-axis (not shown). Coil 30 may comprise a plurality of saddle coils (not shown). Coil 30 may be disposed a predetermined distance away from coil 28. In particular, a gap (G2) may be defined between coils 28, 30. A conductive compound layer 32 may be disclosed in the gap (G2) which will be explained in greater detail below.

Conductive compound layers 30, 32 are provided to minimize the build up of an electric field, E. between adjacent gradient coils to minimize any electrostatic discharges in air bubbles contained within layers 30, 32. Conductive compound layers 30, 32 may be formed from a potting compound. For example, conductive compound layers 30, 32 may be formed from a glue such as an epoxy resin (e.g., bisphenol-A resin) and a chemical hardener such as an anhydride hardener. Alternately, conductive compound layers 30, 32 may be formed from another type of glue, such as a polyester resin. Further, conductive compound layers 30, 32 may contain a plurality of conductive particles such as one of carbon particles, silver particles, copper particles, or gold particles, for example. Further, each of the plurality of conductive particles may have a diameter within the range of 1 to 10 μm. Further, the plurality of conductive particles are preferably disbursed substantially uniformly throughout layers 30, 32. The volume percentage of conductive particles to resin plus hardener is preferably 0.1% or less of conductive particle volume to 99.0% or greater of resin plus hardener volume. The conductive compound layers 30,32 preferably limit a current flowing through layers 30,32 to less then 10 microamps.

Outer gradient coil assembly 25 includes an outer gradient tube 26, an outer (Z) coil 40, an outer (Y) coil 42, an outer (X) coil 44, and conductive compound layers 46, 48.

Outer gradient tube 26 is disposed around an inner gradient tube 24 and is disposed about axis 31. Gradient tube 26 may be constructed from a fiber composite material comprising one or more layers wherein each layer comprises a plurality of fibers such as glass fibers, carbon fibers, Kevlar fibers, and aluminum oxide fibers, coated with the epoxy resin.

Outer (Z) coil 40 is provided to generate a magnetic field gradient along the Z-axis that is disposed to provide electromagnetic shielding of the inner (Z) coil 27 such that any magnetic flux straying outside of the complete gradient coil assembly 22 is minimized. Coil 40 may be disposed proximate gradient tube 26. In particular, coil 40 may be disposed on an outer surface of tube 26. Coil 40 may include a plurality of copper conductors 52 disposed in a plurality of grooves 50 formed in tube 26.

Outer (Y) coil 42 is provided to generate a magnetic field gradient along the Y-axis that is disposed to provide electromagnetic shielding of the inner (Y) coil 28 such that any magnetic flux straying outside of the complete gradient coil assembly 22 is minimized. Coil 42 may comprise a plurality a saddle coils (not shown). Coil 42 may be disposed a predetermined distance away from coil 40. In particular, a gap (G3) may be defined between coils 40, 42. A conductive compound layer 46 may be disposed in the gap (G3) which will be explained in greater detail below.

Outer (X) coil 44 is provided to generate a magnetic field gradient along the X-axis that is disposed to provide electromagnetic shielding of the inner (X) coil 30 such that any magnetic flux straying outside of the complete gradient coil assembly 22 is minimized. Coil 44 may comprise a plurality of saddle coils (not shown). Coil 44 may be disposed a predetermined distance away from coil 42. In particular, a gap (G4) may be defined between coils 42, 44. A conductive compound layer 48 may be disclosed in the gap (G4) which will be explained in greater detail below.

Conductive compound layers 46, 48 are provided to minimize a voltage potential between adjacent gradient coils to minimize any static electricity discharges in air bubbles contained within conductive compound layers 46, 48. Conductive compound layers 46, 48 may be formed from the same types of materials as used for conductive compound layers 30, 32 and have similar current characteristics.

Epoxy layer 54 may be disposed between inner gradient coil assembly 23 and outer gradient coil assembly 25 to maintain a predetermined distance between assemblies 23, 25. Epoxy layer 54 may be formed from an epoxy resin or a polyester resin. Epoxy layer 54 is preferably substantially non-conductive.

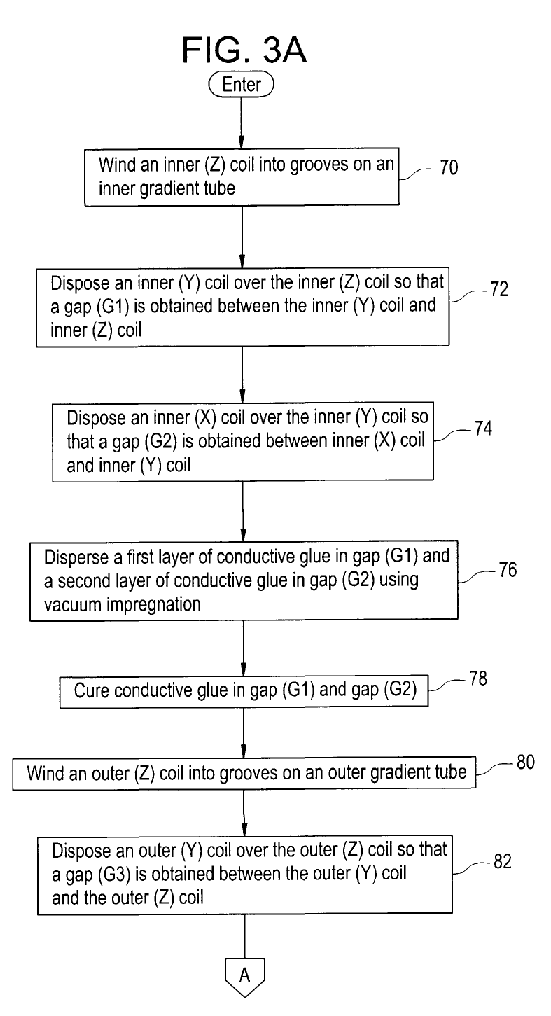

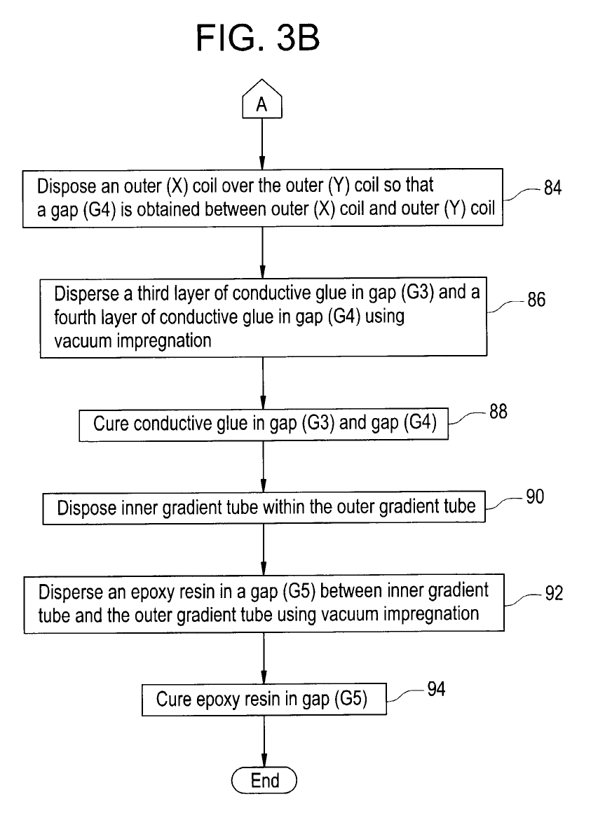

Referring to FIG. 3, an exemplary method for assembling gradient coil assembly 22 will now be explained. At step 70, inner (Z) coil 27 is affixed to inner gradient tube 23.

Next at step 72, inner (Y) coil 28 is disposed over inner (Z) coil 27 so that a gap (G1) is obtained between coils 27, 28.

Next at step 74, inner (X) coil 30 is disposed over inner (Y) coil 28 so that a gap (G2) is obtained between coils 28, 30.

Next at step 76, conductive compound layer 30 is disposed between coils 27, 28 and conductive compound layer 32 is disposed between coils 28, 30. The conductive compound layers 30, 32 may be disposed in gaps (G1), (G2) using vacuum impregnation as known to those skilled in the art.

Next at step 78, conductive compound layers 30, 32 are allowed to cure for predetermined amount of time.

Next at step 80, outer (Z) coil 40 is affixed to outer gradient tube 26.

Next at step 82, outer (Y) coil 42 is disposed over outer (Z) coil 40 so that a gap (G3) is obtained between coils 40, 42.

Next at step 84, outer (X) coil 44 is disposed over outer (Y) coil 42 so that a gap (G4) is obtained between coils 42, 44.

Next at step 86, conductive compound layer 46 is disposed between coils 40, 42 and conductive compound layer 48 is disposed between coils 42, 44. Conductive compound layers 46, 48 may be disbursed in gaps (G3), (G4) using vacuum impregnation as known to those skilled in the art.

Next at step 88, conductive compound layers 46, 48 are allowed to cure for a predetermined amount of time.

Next at step 90, inner gradient tube 24 is disposed within outer gradient tube 26.

Next at step 92, and epoxy resin 54 is disbursed in a gap between inner gradient tube 24 and outer gradient tube 26 using vacuum impregnation as known to those skilled in the art.

Next at step 94, the epoxy resin in gap 54 is allowed to cure.

It should be noted that the order of the steps disclosed in the foregoing method for assembling gradient coil assembly 22 could be switched in order or modified as known to those skilled in the art.

The gradient coil assembly and method related thereto provides a substantial advantage over known assemblies and methods. In particular, the inventive gradient coil assembly utilizes a conductive compound disposed between adjacent gradient coils to minimize the build-up of electric field, E, across a voltage potential in air bubbles contained within the potting compound. Accordingly, undesirable electrostatic discharges within the air bubbles are reduced and/or minimized.

While the invention is described with reference to an exemplary embodiment, it will be understood by those skilled in the art that various changes may be made an equivalence may be substituted for elements thereof without departing from the scope of the invention. In addition, many modifications may be made to the teachings of the invention to adapt to a particular situation without departing from the scope thereof. Therefore, is intended that the invention not be limited the embodiment disclosed for carrying out this invention, but that the invention includes all embodiments falling with the scope of the intended claims. Moreover, the use of the term's first, second, etc. does not denote any order of importance, but rather the term's first, second, etc. are us are used to distinguish one element from another.

1. A gradient coil assembly for use in an MRI device, comprising:
a gradient tube extending along an axis, the tube including first and second gradient coils and a conductive compound disposed between the first and second gradient coils.

2. The gradient coil assembly of claim 1 wherein the conductive compound comprises an epoxy resin having a plurality of conductive particles.

3. The gradient coil assembly of claim 2 wherein the conductive particles comprise one of carbon particles, silver particles, copper particles, and gold particles.

4. The gradient coil assembly of claim 2 wherein the conductive compound further includes a chemical hardening compound.

5. The gradient coil assembly of claim 2 wherein the epoxy resin comprises a bisphenol-A resin.

6. The gradient coil assembly of claim 1 wherein the conductive compound comprises a polyester resin having a plurality of conductive particles.

7. The gradient coil assembly of claim 6 wherein the conductive particles comprise one of carbon particles, silver particles, copper particles, and gold particles.

8. The gradient coil assembly of claim 6 wherein the compound further includes a chemical hardening compound.

9. The gradient coil assembly of claim 1 wherein each of the conductive particles is less than 10 μm in diameter.

10. The gradient coil assembly of claim 1 wherein the conductive compound limits current flow through the compound to less than a predetermined current value.

11. The gradient coil assembly of claim 10 wherein the predetermined current value is 10 microamps.

12. A gradient coil assembly for use in an MRI device, comprising:
a gradient tube extending along an axis, the tube including first and second gradient coils and a potting compound layer of disposed between the first and second gradient coils, the potting compound layer having a plurality of conductive particles configured to limit a current flowing through the compound layer to less than a predetermined current value.

13. A method for assembling a gradient coil assembly, comprising:
disposing a first gradient coil on a first gradient tube; and,
disposing a conductive compound between the first gradient coil and a second gradient coil.

14. The method of claim 13 wherein the disposing a conductive compound layer includes vacuum impregnating the conductive compound between the first and second gradient coils.

15. The method of claim 13 wherein the conductive compound comprises an epoxy resin having a plurality of conductive particles.

16. The method of claim 15 wherein the conductive compound further includes a chemical hardening compound.

17. The method of claim 15 wherein the conductive particles comprise one of carbon particles, silver particles, copper particles, and gold particles.

18. The method of claim 13 wherein the conductive compound comprises a polyester resin having a plurality of conductive particles.

19. The method of claim 13 wherein the conductive compound limits current flow through the layer to less than a predetermined current value.

20. The method of claim 19 wherein the predetermined current value is 10 microamps.
